(19) United States
(12) Patent Application Publication
(10) Pub. No.: US 2025/0044229 A1
(43) Pub. Date: Feb. 6, 2025

(54) CALIBRATION STANDARD, SENSOR ARRANGEMENT AND USE

(71) Applicant: Endress+Hauser Conducta GmbH+Co. KG, Gerlingen (DE)

(72) Inventors: Andreas Bayer, Gerlingen (DE); Thilo Krätschmer, Gerlingen (DE); Felicia Seichter, Ludwigsburg (DE); Joachim Bolle, Stockach (DE)

(21) Appl. No.: 18/719,957

(22) PCT Filed: Nov. 21, 2022
(86) PCT No.: PCT/EP2022/082670

§ 371 Date: Jun. 14, 2024

(30) Foreign Application Priority Data
Dec. 16, 2021 (DE) 10 2021 133 467.5

(51) Int. Cl. G01N 21/64

(52) U.S. Cl. CPC G01N 21/645; G01N 2021/6463; G01N 2201/127; G01N 2201/13

(57) ABSTRACT

The invention discloses a calibration attachment for adjustment, calibration and/or for carrying out a functional check of an optical sensor, which is configured for measuring at least one measurement variable in a medium using light. The sensor is configured for emitting emission light of at least a wavelength in the range of 200-450 nm, comprising a housing and a body arranged in the housing wherein the body comprises praseodymium, and after excitation with the emission light, the body emits light with a longer wavelength. The invention also discloses a sensor arrangement comprising such a calibration attachment, and a use of same.

The invention relates to a calibration standard for adjusting, calibrating, and/or for carrying out a functional check of an optical sensor, to a sensor arrangement, and to a use.

The sensor is a fluorescence sensor. In fluorescence measurement, the medium is generally irradiated with a shortwave excitation light (transmitted light) and the longer-wave fluorescent light (received light) that is generated by the medium and that is generated as a function of the concentration of the species to be measured is detected. The fluorescence sensor comprises a light source and a receiver. The light source transmits transmitted light; the receiver receives received light.

Such a fluorescence sensor must, at regular intervals, be subjected to a functional test and/or calibrated. For this purpose, either corresponding standard liquids or else solids with defined fluorescence can be used in liquid measuring devices. It is of central importance here that the emitted fluorescence intensity of the standard is stable against environmental influences, aging and the exciting UV radiation.

In the case of liquid standards, aging can be observed very quickly; a clear change in the fluorescence intensity results after a few days already. In addition, the handling of liquids is cumbersome.

Solid standards available on the market often contain organic material, e.g., on a PMMA basis. This results in a strong degradation of the fluorescence signal, which is caused by the exciting UV radiation.

Temperature stability is also necessary, which the above-described standards do not fulfill.

The object of the invention is to overcome the described disadvantages. In particular, in the case of optical sensors, it should be possible to carry out universal and complete calibration with long-term stable calibration means.

The object is achieved by a calibration attachment for adjusting, calibrating, and/or for carrying out a functional check of an optical sensor, which is designed to measure at least one measured variable in a medium by means of light, wherein the sensor is designed to emit transmitted light at least of a wavelength in the range of 200-450 nm, comprising a housing and a body, which is arranged in the housing, wherein the body comprises praseodymium, and wherein the body, after excitation with the transmitted light, in particular by absorption of the transmitted light, emits light of a different, in particular longer, wavelength.

The sensor is, for example, a fluorescence sensor, wherein fluorescence can be defined as the emission of light of a wavelength that results from the absorption of light of a different, in particular shorter, wavelength.

For this purpose, the praseodymium, which the body comprises, must be located at the surface of the body or the body itself is substantially transparent to the excitation wavelength, i.e., the transmitted light.

For checking measurement accuracy, the functions, and for the possibility of recalibration or adjustment of fluorescence sensors, in the present case, a solid standard was invented, which provides a suitable fluorescence measurement value, which is stable to a significantly higher degree against aging, environmental influences, and the excitation radiation than previously available concepts.

The object addressed is achieved by using praseodymium. A sufficiently strong fluorescence signal in the wavelength range of 250-500 nm, in particular 340-380 nm is generated. Due to use in the UV range with transmitted light of 200-450 nm, stability against the exciting UV light is particularly critical, which is less important in other wavelength ranges, such as the infrared wavelength range.

As has been found when using praseodymium, the fluorescence intensity in the measurement range does not change at all or changes very little under the following influences:
Multi-day irradiation with the excitation light of the fluorescence measuring device
Temperature changes within the range of −10° C. to +80° C.
Humidity changes
Storage over several years

As mentioned, the special properties result with praseodymium. In the case of reduced stability criteria, a body comprising cerium, silver, lead, cobalt, manganese, nickel, neodymium, samarium or zinc can also be used.

However, the best results are obtained with praseodymium.

One embodiment provides that the body is a glass body.

One embodiment provides that the glass body is doped with praseodymium.

One embodiment provides that praseodymium or a praseodymium compound, in particular praseodymium oxide, is added to a glass-forming material, and a glass-transforming material, and this mixture is heated to form glass in order to obtain the glass body by solidification.

One embodiment provides that the glass body is doped by ion exchange.

One embodiment provides that praseodymium or a praseodymium compound, in particular praseodymium oxide is added to a glass melt as particles, crystals, agglomerates or the like, in particular praseodymium or the praseodymium compound is sintered into the glass melt, or praseodymium or a praseodymium compound is further melted down into the glass melt.

One embodiment provides that glass bodies are produced by fusing two glasses, one of the two being a non-coloring glass and the other colored glass, comprising praseodymium or a praseodymium compound, in particular praseodymium oxide.

One embodiment provides that the glass body comprises a glass-ceramic and/or partially crystalline material.

One embodiment provides that the body is a plastics body.

One embodiment provides that a praseodymium-containing compound is added to the glass or plastics body; in one embodiment, the claimed praseodymium therefore also comprises praseodymium-containing compounds.

One embodiment provides that the body is mounted in the housing via a mechanical holder.

One embodiment provides that the body is designed to be disk-shaped or lens-shaped.

For use as a solid standard, a sample of the relevant glass in the form of either a disk or a lens-like bead is placed in a mechanical mount, which can be attached to the sensor.

One embodiment provides that the housing comprises a receptacle for the sensor.

One embodiment provides that the housing is substantially transparent to the transmitted light.

One embodiment provides that the housing comprises an opening and transmitted light impinges on the body through the opening.

The object is furthermore achieved by a sensor arrangement comprising at least one light source, wherein the light source emits transmitted light at least of a wavelength in the range of 200-450 nm; at least one receiver, which is designed to receive received light of a greater wavelength than the transmitted light, in particular of a wavelength of 250-500 nm; and a calibration attachment as described above, wherein the light emitted by the body forms the received light.

The object is furthermore achieved by the use of praseodymium for adjusting, calibrating, and/or for carrying out a functional check of an optical sensor, wherein the sensor is designed to emit transmitted light at least of a wavelength in the range of 200-450 nm.

This is explained in more detail with reference to the following drawings.

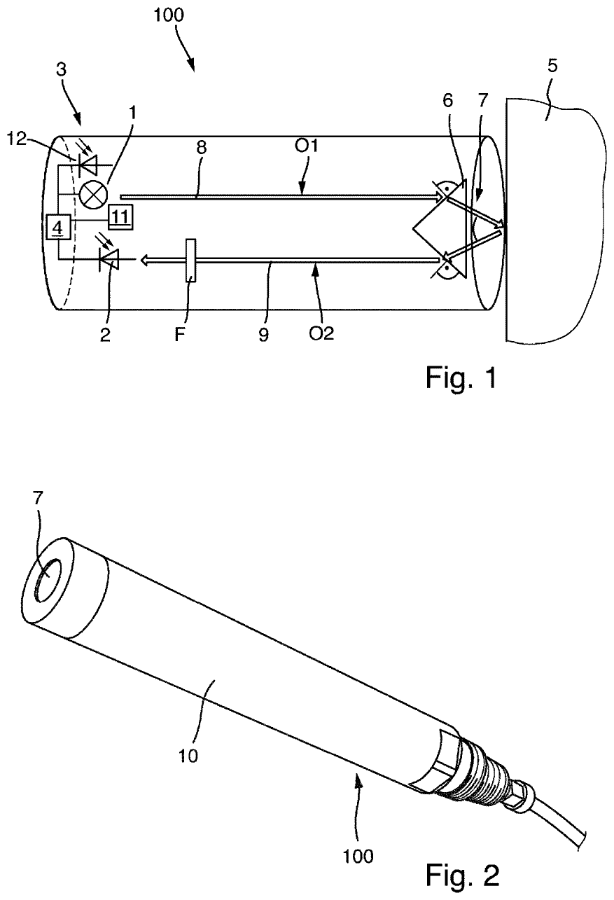

FIG. 1 shows the claimed sensor in a symbolic cross section.

FIG. 2 shows the claimed sensor.

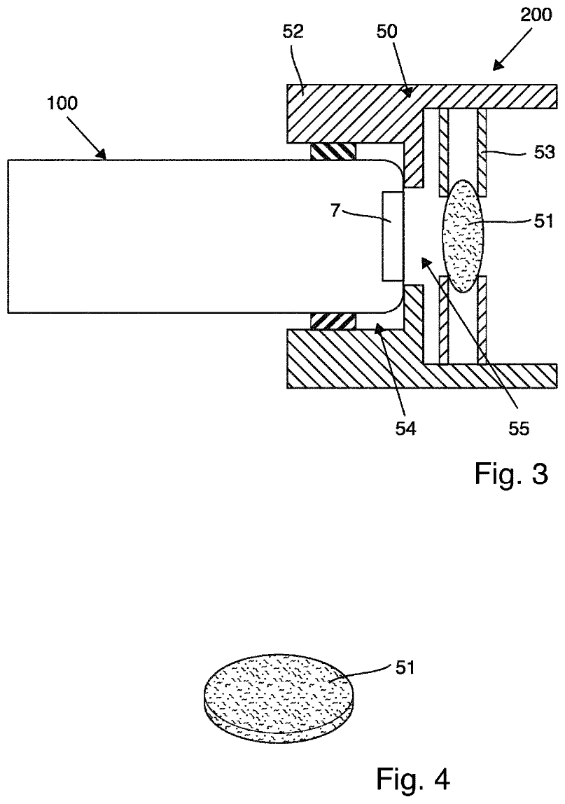

FIG. 3 shows a cross section through the calibration attachment.

FIG. 4 shows the body comprising praseodymium.

In the drawings, the same features are labeled with the same reference signs.

The claimed calibration attachment 50 is suitable for adjusting, calibrating, and/or for carrying out a functional check of an optical sensor 100, which is designed to measure at least one measured variable in a medium 5 by means of light, wherein “light” is transmitted light or received light (see below). The sensor is a fluorescence sensor, which is to be discussed first. The sensor in its entirety is denoted by reference sign 100 and is shown schematically in FIG. 1. FIG. 2 shows the sensor 100 with its housing 10 and an optical window 7.

In principle, the sensor 100 is suitable for determining the oil-in-water content of a medium 5 or for determining the PAH content during flue gas scrubbing, for example on ships. Other applications are however possible. Mention should be made here, for example, of the measurement of acetylsalicylic acid or use in food analysis, e.g., of vitamins, or linoleic acid or material differentiation by means of fluorescence markers.

FIG. 1 shows the sensor 100 during measurement operation. The calibration operation is discussed below with reference to FIG. 3.

A light source 1 transmits transmitted light 8 toward the medium 5. The light source 1 is, for example, an LED which emits light of a wavelength of 200-450 nm, e.g., 255 nm. It is also possible to use a laser as the light source, or Xenon or mercury gas discharge lamps (254 nm), optionally with corresponding frequency filters.

The sensor 100 comprises a data processing unit 4, e.g., a microcontroller. The data processing unit 4 controls the light source 1 to transmit transmitted light 8 toward the medium 5 (measurement operation) or calibration attachment 50 (calibration operation, FIG. 3). The LED 1 is, for example, operated with a settable current source. The amplitude of the transmitted light is approximately proportional to the operating current of the LED 1.

The transmitted light 8 impinges on a prism 6 at an angle. The prism 6 is a right-angled prism, for example. The base points toward the medium 5 to be measured. A first optical path from the light source 1 to the prism 6 results. The optical path may also contain one or more lenses or filters.

The transmitted light 8 is partially converted into received light 9 in the medium 5 by fluorescence as a function of the concentration of the substance to be measured in the medium 5. The received light 9 takes the path toward the receiver 2 via the prism 6.

The receiver 2 is a photodiode, which receives the received light 9 at a wavelength of 300-400 nm. The filter F in FIG. 1 filters, for example, for wavelengths of 340-380 nm. In principle, the receiver 9 is able to measure in a broader range, e.g., from 190-1100 nm. A second optical path from the prism 6 to the receiver 2 results. The optical path may also contain one or more lenses or filters. The first and second optical paths are substantially parallel to one another on the side of the prism facing away from the medium.

The sensor 100 comprises a monitor diode 12, which monitors the transmission power of the LED 1.

The sensor 100 comprises a temperature sensor 11, which measures the temperature of the light source 1.

The light source 1, prism 6, and receiver 2 are arranged in a housing 10. The housing is tube-shaped, with a diameter of 35-75 mm. The housing 10 comprises an optical window 7, which is permeable at least to transmitted light 8 and received light 9, wherein the prism 6 and the window 7 are either cemented, glued, joined together, or manufactured from one piece. In one embodiment, the individual components are separate. The distance from the light source 1 or the receiver 2 to the window 7 is approximately 2-6 cm.

The filter(s) are designed as wavelength filters, e.g., as interference filters.

FIG. 1 describes the sensor 100 in measurement operation. With reference to FIG. 3, the calibration operation with the calibration attachment 50 is now discussed, wherein, from an optical point of view, the medium 5 is only exchanged with the calibration attachment 50 or body 51.

By means of the calibration attachment 50, the optical sensor 100 can be adjusted, calibrated, and/or a functional check can be carried out. The calibration attachment 50 has a housing 52, which is manufactured from plastic, for example. In principle, the calibration attachment 50 can also be manufactured from a metal such as aluminum or from stainless steel.

The housing 52 has a receptacle 54 for the sensor 100. Thereby, the sensor 100 reaches the correct location and the transmitted light or received light can reach the body 51 via the optical paths from the light source 1. For this purpose, the housing 52 has an opening 55. In principle, a variant without an opening is also possible; the housing 52 must then be transparent to the corresponding wavelengths of the light source 1 or after conversion.

The body 51 is arranged in the interior of the housing 52, wherein the body 51 is fastened via a mechanical holder 53. The body 51 comprises praseodymium.

Alternatively, cerium, silver, lead, cobalt, manganese, nickel, neodymium, samarium, or zinc can be used. However, praseodymium showed the best results.

The body 51 comprising praseodymium emits light of a different, in particular longer, wavelength after excitation with the transmitted light, in particular by absorption of the transmitted light.

The body 51 is, for example, designed as a glass body. The glass body is made of barium phosphate glass or a quartz glass, for example. The glass body is doped with the praseodymium. In one embodiment, the body is made of plastics.

In general, the body is transparent to the emission wavelengths used and to the light converted by fluorescence.

The body 51 is designed to be disk-shaped or lens-shaped. However, the basic concept of the present invention also works with fragments or using a part having any shape.

FIG. 3 thus shows the sensor arrangement 200 comprising the sensor 100 and the calibration attachment 50.

FIG. 4 shows the body 51 with praseodymium.

### LIST OF REFERENCE SIGNS

1 Light source
2 Receiver
4 Data processing unit
5 Medium
6 Prism
7 Optical window
8 Transmitted light
9 Received light
10 Housing
11 Temperature sensor
12 Monitor diode
50 Calibration attachment
51 Body
52 Housing
53 Mechanical holder
54 Receptacle
55 Opening
100 Sensor
200 Sensor arrangement
F Filter

1-11. (canceled)

12. A calibration attachment for adjusting, calibrating, or carrying out a functional check of an optical sensor, which is designed to measure at least one measured variable in a medium by means of light, wherein the sensor is designed to emit transmitted light at least of a wavelength in the range of 200-450 nm, comprising:
a housing; and
a body, which is arranged in the housing;
wherein the body comprises praseodymium; and
wherein the body, after excitation with the transmitted light;
emits light of a longer wavelength.

13. The calibration attachment according to claim 12,
wherein the body is a glass body.

14. The calibration attachment according to claim 13,
wherein the glass body is doped with praseodymium.

15. The calibration attachment according to claim 12,
wherein the body is a plastics body.

16. The calibration attachment according to claim 12,
wherein the body is mounted in the housing via a mechanical holder.

17. The calibration attachment according to claim 12,
wherein the body is disk-shaped or lens-shaped.

18. The calibration attachment according to 12,
wherein the housing comprises a receptacle for the sensor.

19. The calibration attachment according to 12,
wherein the housing is substantially transparent to the transmitted light.

20. The calibration attachment according to 12,
wherein the housing comprises an opening and transmitted light impinges on the body through the opening.

21. A sensor arrangement, comprising:
a sensor having:
at least one light source, wherein the light source emits transmitted light at least of a wavelength in the range of 200-450 nm;
at least one receiver, which is designed to receive received light of a wavelength of 250-500 nm; and
a calibration attachment;
wherein the calibration attachment includes:
a housing; and
a body, which is arranged in the housing;
wherein the body comprises praseodymium; and
wherein the body, after excitation with the transmitted light, emits light of a longer wavelength;
wherein the light emitted by the body forms the received light.

22. A method of adjusting, calibrating or carrying out a functional check of an optical sensor, including steps of:
emitting transmitted light at least of a wavelength in the range of 200-450 nm using a sensor, wherein the sensor is designed to measure at least one measured variable in a medium using light;
exciting a calibration attachment of the optical sensor with the transmitted light, wherein the calibration attachment includes a housing and a body arranged in the housing, wherein the body comprises praseodymium;
upon excitation, the body emitting light of a longer wavelength; and
adjusting, calibrating or carrying out a functional check of the optical sensor.
